SEQUENCE LISTING (23 sequences)

SEQ ID NO: 1 - DNA, 873 bp, Nicotiana benthamiana
atgggaaatgctaactgtgtattttgtggatgcatagaacaagcgagcgttggtgtggttgagaaatggggacgttttgacaggcttgcagaaccggggcttaacttcttcaatcctttagccggcgaatgcctctctggtattctctccaccaggatcagttctctcgatgtcaaaatcgagactaaaaccaaggacaatgtctttgttcatttagtgtgctcgatccaatatagagtgatcaggcaaaatgctgatgatgctttctatgagttgcaaaatccgaaggagcagattcaggcttatgtatttgatgttgttcgagcccatgtccccaaaatgaatttggatgaacttttcgagcaaaaggatgaagttgctaaggctgtgttggaggaacttgagaaggtgatgggtgcgtatggatataacatcgagcacatactgatggttgacattattcctgatgcttctgtacgaaaggcaatgaacgagataaatgcagctcaaaggatgcagcttgctagtgtatacaagggagaagcagaaaagattctccaagttaagaaagcagaagctgaggctgaagccaagtatttaggtggggttggggttgctaggcagaggcaggcaattacagatggtttgagagaaaacatcttgaacttttcacataaagtagaaggtacctccgctaaagaagtgatggatcttataatgatcacccagtactttgacaccatcaaagaccttggaaactcttcaaagaacacgacagttttcataccacatggtcctggccatgttcgtgacattggtgatcagatacgcaacggcctgatggaggcagctagtgcacaggtcgctgaatag

SEQ ID NO: 2 - DNA, 873 bp, Nicotiana benthamiana
atggggaatgctaattgtgtattctgtggatgcatagaacaagcgagcgtcggtgtggttgagaaatggggacgttttgacaggctagcagaaccggggcttaacttcttcaatcctttcgccggcgaatgcctttctggtattctctccaccaggatcagttctctcgatgtcaaaatcgagactaaaaccaaggacaatgtctttgttcatttagtgtgctcgatccaatatagagtgatcaagcaaaatgctgatgatgctttctatgagttgcaaaatccaaaggagcagattcaggcttatgtatttgatgttgttcgtgcccatgtccccaaaatgaatttggatgaacttttcgagcaaaaggatgaagttgctaaggctgtgttggaggaacttgagaaggtaatgggtgcatatggatataacatcgagcacatactgatggttgacattattcctgatgcttctgtgcgaaaggcgatgaacgagataaatgcagctcaaaggatgcagcttgctagtgtatacaagggagaagcagaaaagattctccaagttaagaaagcagaagctgaggctgaagccaagtatttaggtggggttggggttgccaggcagagacaggcaattacagatggtttgagagaaaacatcttgaacttttcacataaagtagaaggcacctctgctaaagaagtgatggatcttataatgatcacccagtactttgacaccatcaaagaccttggaaactcttcaaagaacacgacagttttcataccacaaggtcctgggcatgttcgtgacattggtgatcagatacgcaacggcctgatggaggcagctagtgcacaggtcactgaatag

SEQ ID NO: 3 - DNA, 25 bp, Artificial sequence
  primer
atggaaatgctaactgtgtattgtg

SEQ ID NO: 4 - DNA, 24 bp, Artificial sequence
  primer
ctattcagcgacctgcactagctg

SEQ ID NO: 5 - DNA, 25 bp, Artificial sequence
  primer
atgggaatgctaattgtgtattctg

SEQ ID NO: 6 - DNA, 24 bp, Artificial sequence
  primer
ctattcagtgacctgcactagctg

SEQ ID NO: 7 - DNA, 28 bp, Artificial sequence
  primer
atgggaaatgctaactgtgtattttgtg

SEQ ID NO: 8 - DNA, 26 bp, Artificial sequence
  primer
ctattcagcgacctgtgcactagctg

SEQ ID NO: 9 - DNA, 26 bp, Artificial sequence
  primer
atggggaatgctaattgtgtattctg

SEQ ID NO: 10 - DNA, 26 bp, Artificial sequence
  primer
ctattcagtgacctgtgcactagctg

SEQ ID NO: 11 - DNA, 21 bp, Artificial sequence
  primer
aggagcagattcaggcttatg

SEQ ID NO: 12 - DNA, 22 bp, Artificial sequence
  primer
ccacctaaatacttggcttcag

SEQ ID NO: 13 - DNA, 22 bp, Artificial sequence
  This sequence is a primer sequence and it is synthesized
aggagcagattcagggcttatg

SEQ ID NO: 14 - DNA, 20 bp, Artificial sequence
  primer
gataatcatcgcaagaccgg

SEQ ID NO: 15 - DNA, 20 bp, Artificial sequence
  primer
aagaccagctcatccgtgga

SEQ ID NO: 16 - DNA, 21 bp, Artificial sequence
  primer
ctcatcctatcagcaatgccc

SEQ ID NO: 17 - DNA, 22 bp, Artificial sequence
  primer
tggaaatgggaaactggtggtc

SEQ ID NO: 18 - DNA, 27 bp, Artificial sequence
  primer
gacaagggaatatcggtaagattattg

SEQ ID NO: 19 - DNA, 22 bp, Artificial sequence
  primer
gaaacgcctatcggaaacactg

SEQ ID NO: 20 - DNA, 25 bp, Artificial sequence
  primer
aagccaatacactcattacagcatc

SEQ ID NO: 21 - DNA, 20 bp, Artificial sequence
  primer
acaagactatttggatgccc

SEQ ID NO: 22 - DNA, 19 bp, Artificial sequence
  primer
tctcaacagccttagcagc

SEQ ID NO: 23 - DNA, 867 bp, Oryza sativa L. ssp. Japonica. cv.
atggtgagcgccttcttcctgctgtgcgggtgcgtggaccaggcgagcgtcgcggtggtggagaagtggggccgcttcctccgcctcgccgagccgggcctccacttcttcaacccgttcgccggcgagttcgtcgccgggacgctctccacccgcgtccagtcgctcgacgtccgcgtcgagaccaagaccaaggataatgtctttgttcagcttatctgcacaatccaatatcgggttgttaaggaacatgctgatgatgcattctatgagttgcagaatccccaacagcaaattcaggcctacgtctttgatgtggtccgagctatagttccgagaatgaatcttgatgatctttttgagcaaaagaatgatgtggcgaaagctgtacttcaggagctagaaaaggtgatgggagattatggttacagcattgagcacattctcatggttgacatcatccctgatgctgctgtacgcagagcaatgaatgaaataaatgcagcacaaaggcttcagcttgcaagtgtctacaaaggagaggcggagaagattcttctggtgaagaaagcagaagcagaggcagaggcaaaacacctttccggtgtcggcattgctagacagcggcaggcgataactgatggcctgagagagaacatcctgaacttctcgcactcggtttcaggcacctcagcaaaagaagtcatggatctcatcatggtcacgcagtacttcgacaccatcaaagaacttggggatggctcgaagaacaccacggtgttcatacctcatggcccaggccatgtcagggatatcagcgagcaaatccggaatggtatgatggaagcgtcctgcagcaacgtgtaa

(19) United States
(12) Patent Application Publication
(10) Pub. No.: US 2020/0216855 A1
(43) Pub. Date: Jul. 9, 2020

(54) Disease Resistant Plants Containing HIR3 Gene and Method for making the plants thereof

(71) Applicant: Ningbo University, Ningbo (CN)

(72) Inventors: Fei YAN, NINGBO (CN); Saisai LI, NINGBO (CN); Yuwen LU, NINGBO (CN); Jinping ZHAO, NINGBO (CN); Jiejun PENG, NINGBO (CN); Hongying ZHENG, NINGBO (CN); Lin LIN, NINGBO (CN); Ye CHENG, NINGBO (CN); Jianping CHEN, NINGBO (CN)

(21) Appl. No.: 16/523,703

(22) Filed: Jul. 26, 2019

(30) Foreign Application Priority Data
Jan. 8, 2019 (CN) 201910016241.8
Jan. 8, 2019 (CN) 201910016244.1

(51) Int. Cl. C12N 15/82; C07K 14/415

(52) U.S. Cl. CPC C12N 15/8283; C07K 14/415

(57) ABSTRACT

The present invention provide the use of Nicotiana benthamiana (N. benthamiana) HIR3s gene and/or Oryza sativa HIR3 gene in producing plants with resistance to virus and the method for making the plants thereof, the method involve: constructing NbHIR3.1, NbHIR3.2 or OsHIR3 into plant binary expression vector pCV1300 respectively, and introduced into Agrobacterium by electric shock, then transgenic plants overexpressing either NbHIR3.1 or NbHIR3.2 gene or tobacco or rice overexpressing HIR3 were produced by infection with Agrobacterium; the nucleotide sequences of NbHIR3.1, NbHIR3.2 and OsHIR3 are shown as SEQ ID NO:1, SEQ ID NO:2 and SEQ ID NO:23 respectively.

### CROSS REFERENCE TO RELATED APPLICATION

The present application claims the benefit of Chinese Patent Application No. 201910016244.1, filed on Jan. 8, 2019, and Chinese Patent Application No. 201910016241.8, filed on Jan. 8, 2019. The content of these applications including all tables, diagrams and claims is incorporated hereby as reference in its entity.

### FIELD OF THE INVENTION

The invention is related to the field of genetic engineering technology and plant disease control, in particular to the application of transgenic plants over expressing HIR3 gene in plant basic resistance.

### BACKGROUND OF THE INVENTION

In the natural environment, the invasion of pathogens often occurs in the whole process of plant growth and development. In many cases, plants can normally grow, develop and reproduce. In the long-term process of interaction evolution, plants have developed a series of defense systems against pathogen infection. Defense network against pathogens of plant is a complex and delicate system, including identification of host and pathogen, signal transduction and regulation of defense genes and many other processes.

The hypersensitive response (HR) is a crucial component of the plant immune system to limit the spread of pathogenic infection. The main feature of HR is the rapid death of the surrounding cells, subsequently inducing local hypersensitive response (i.e. blocking the damage sites) to limit pathogenic further infection. The hypersensitive induced reaction gene family is associated with HR and participates in plant defense against pathogens. The HIR family gene products are homologs of the HR-inducing protein NG1 of Nicotiana tabacum. NG1 is an activator of HR and its over-expression results in the formation of HR-like lesions. The first homologs of NG1 identified from a different plant species are three proteins from maize, named ZmHIR1, ZmHIR2 and ZmHIR3. Based on their DNA and amino acid similarities to maize HIR genes, four HIR genes in barley (Hordeum vulgare ssp. vulgare L.) were then isolated and named HvHIR1-4. Fast-neutron mutants of barley exhibiting spontaneous HR (disease lesion mimic mutants) on their leaves had up to a 35-fold increase in HvHIR3 expression, implicating HIR genes in the induction of HR. After that, HIRs were identified in a legume (Lotus japonicus), cucumber, rice and wheat and were shown to be associated with microspore development and plant responses to bacterial infection.

Previous studies focus on HIR1, but few on other HIRs. Transgenic Arabidopsis thaliana overexpressing rice OsHIR1 show resistance to Pseudomonas syringae (Pst. DC3000). It is not clear whether HIR3s, similar to other members of the HIR family, can induce HR and participate in plant defense against pathogens.

During the long-term evolution process, host plants have formed a series of complex and delicate HR signal transduction mechanisms through sensing signals, signal transduction and inducing the expression of defense genes, in order to adapt to environmental stimuli and effectively reduce the damage caused by biotic and abiotic stresses. Ca2+ ions, reactive oxygen species, hormones and other signaling molecules play important roles in the signal transduction of HR. Salicylic acid (SA), jasmonic acid (JA) and ethylene (ETH) are involved in HR signal transduction, the important roles of which in plant defense response have been widely studied and applied.

At present, the research on HIR mainly focuses on HIR 1, and there are many studies on the role of HIRs in plant resistance to bacterial and fungal pathogens. There are few studies on the functions of other HIRs, and it is not clear whether HIRs are involved in the process of Plant Anti-virus infection.

Although the defense roles of HIR1 against bacterial and fungal pathogens have been well-studied, little is known about other HIRs and how HIRs respond to viral infection.

### SUMMARY OF THE INVENTION

It was found that the infection of Rice stripe virus (RSV) induced up-regulating expression of HIR3 genes in host Nicotiana benthamiana (N. benthamiana) (the nucleotide sequences of the genes are shown as SEQ ID NO: 1 and SEQ ID NO: 2, respectively).

Two highly homologous HIR3 genes from N. benthamiana, named NbHIR3.1 and NbHIR3.2 were cloned, and transient expression vectors were constructed, respectively. NbHIR3.1 and NbHIR3.2 genes were successfully overexpressed in N. benthamiana by leaf disc method, respectively. All stable transgenic lines of N. benthamiana overexpressing either NbHIR3.1 or NbHIR3.2 developed normally without obviously changed phenotype, compared with wild type (WT) N. benthamiana.

After frictional inoculation with RSV, more slight symptom was observed on transgenic lines of N. benthamiana overexpressing either NbHIR3.1 or NbHIR3.2. At the early stage after inoculated RSV, it flowered normally, without dwarf symptom, RSV RNAs in leaves inoculated with RSV and systemic leaves decreased significantly. Thus, it is possible that NbHIR3s improved the tolerance of plants against RSV infection. In addition, transgenic lines of N. benthamiana overexpressing either NbHIR3.1 or NbHIR3.2 lessened the symptoms of virus infection and reduced the accumulation of virus significantly after inoculating Turnip mosaic virus (TuMV) or Potato virus X (PVX). Transgenic lines of N. benthamiana overexpressing either NbHIR3.1 or NbHIR3.2 had significantly lower bacterial biomass than WT after inoculated with Pseudomonas syringae (Pst. DC3000), which means that NbHIR3s bring high resistance against Pst. DC3000.

In summary, NbHIR3s contribute to N. benthamiana plants basal resistance to RSV, TuMV, PVX and Pst. DC3000, especially confer plant resistance to viral infection.

Therefore, NbHIR3.1 and NbHIR3.2 is overexpressed in N. benthamiana plants in this invention, respectively, and produce the transgenic plants bearing basic resistant to RSV, TuMV, PVX and Pst. DC3000 through resistance identification. This is the first time that the HIR3 gene is used as a basic resistant gene in the genesis of transgenic disease-resistant plants.

Therefore, the first object of this invention is to provide the use of N. benthamiana HIR3s gene in plant defense against virus infection. That is, the use of HIR3s gene from N. benthamiana in plant defense against RSV, TuMV, PVX and Pst. DC3000 infection. On the other hand, it provides the application of HIR3s genes from N. benthamiana in the production of plants resistant to RSV, TuMV, PVX and Pst. DC3000 infection. The transgenic plants overexpressing either NbHIR3.1 or NbHIR3.2 produced by the invention are mainly applied to defense against the infection of RSV, TuMV, PVX and Pst. DC3000, and reduce the damage of these viral and bacterial diseases.

In some embodiments, the nucleic acid sequences described are shown as SEQ ID NO:1 and SEQ ID NO:2.

In some embodiments, the plant is Nicotiana benthamiana (N. benthamiana).

In some embodiments, disease-resistant plants are produced by transferring SEQ ID NO:1 and SEQ ID NO:2 sequences to plants.

In some embodiments, the virus is one or some of Rice stripe virus (RSV), Turnip mosaic virus (TuMV), Potato virus X (PVX).

In some embodiments, genes are transformed or transferred by Agrobacterium tumefaciens-mediated methods.

In the N. benthamiana genome, there are two homologs of HIR3 with 98% nucleotide identity and 99% amino acid identity to one another (named NbHIR3.1 and NbHIR3.2), the nucleotide sequences of which are shown as SEQ ID NO:1 and SEQ ID NO:2, respectively. The HIR3s gene sequences were amplified by common PCR using primers and N. benthamiana cDNA as template. These NbHIR3s were respectively 78% and 73% identical to Arabidopsis HIR3 (AtHIR3, Accession No. At5g51570) and rice HIR3 (OsHIR3, Accession No. 0s06g0136000).

Therefore, this invention provides a method for transgenic plants, including:

The NbHIR3.1 and NbHIR3.2 gene were constructed into plant binary expression vector pCV1300, respectively, and introduced into Agrobacterium strains EHA105 by electric shock. transgenic N. benthamiana overexpressing either NbHIR3.1 or NbHIR3.2 gene were produced by leaf disc method.

In some embodiments, the nucleic acid sequences of NbHIR3.1 and NbHIR3.2 are shown as SEQ ID NO:1 and SEQ ID NO:2 respectively.

In some embodiments, the binary expression vector includes the following structure: LB-35s PolyA-HPTII-35s promoter-Nos-target gene-35s promoter-RB which is shown in FIG. 1.1.

In some embodiments, the original binary expression vector includes the structure shown in FIG. 1C.

In some embodiments, the produced transgenic plants have defense resistance against viral infection. In some embodiments, the produced transgenic plants have defense resistance against bacterial infection. In some embodiments, the viruses are TuMV, PVX and RSV, the bacteria is Pst. DC3000.

In some embodiments, the produced transgenic plants have the function of elevating the level of SA.

In some embodiments, the produced transgenic plants elevate SA content by up-regulating the expression of EDS1, NPR1 and PR1 genes.

On the other hand, we found that RSV infection induced up-regulation expression of OsHIR3 in rice. We cloned OsHIR3 gene from Oryza sativa L. ssp. japonica. cv. Nipponbare and constructed a transient expression vector. OsHIR3 gene was successfully overexpressed in rice by derivative method of rice mature embryos. Transgenic lines of rice overexpressing OsHIR3 developed normally without obviously changed phenotype in seed germination, seedling growth, plant height and seed setting compared with wild type (WT) rice.

Wild type (WT) and transgenic rice plants were inoculated with RSV by allowing viruliferous planthopper vectors to feed on them for three days, and subsequent viral infection was monitored. The symptom of transgenic rice overexpressing OsHIR3 gene was mild and dwarfing was alleviated after RSV infection, while the susceptible plants showed only stripe phenotype, and RSV RNAs was significantly reduced. The results therefore indicate that OsHIR3 in rice also plays key roles against RSV.

Furthermore, transgenic rice plants overexpressing OsHIR3 gene were inoculated with Xanthomonas oryzaepv. oryzae (Xoo), an important bacterial pathogen of rice. Resistance of OsHIR3 transgenic rice to Xoo which can cause bacterial blight, was also detected: three independent transgenic lines overexpressing OsHIR3 had significantly shorter lesions than the controls. It therefore appears that the transgenic plants also gained resistance to this bacterial pathogen.

Therefore, OsHIR3 is overexpressed in rice plants in this invention, and produce the transgenic plants with basic resistance against RSV and Xoo through resistance identification. This is the first time that the HIR3 gene is used as a basic resistant gene in the genesis of transgenic disease-resistant rice.

The first purpose of this invention is to provide OsHIR3 gene.

The second purpose of this invention is to provide the use of OsHIR3 gene.

In order to achieve the first purpose mentioned above, this invention adopted technical proposal as following:

There are six HIR family genes in Oryza sativa L. ssp. japonica. cv. Nipponbare. Sequence analysis showed all HIR belong to HIR1 family, except OS06g0136000, which belongs to HIR3 family, the nucleotide sequence of OsHIR3 gene was shown as SEQ ID NO: 23. The OsHIR3 gene sequence was amplified by common PCR using primers and rice cDNA as template.

In order to achieve the second purpose mentioned above, this invention adopted technical proposal as following:

The OsHIR3 gene was constructed into plant binary expression vector pCV1300, named pCV:OsHIR3, and introduced into Agrobacterium strains EHA105 by electric shock. Transgenic rice plants overexpressing OsHIR3 gene were produced by derivative method of rice mature embryos.

RSV-tolerant transgenic rice overexpressing OsHIR3 gene was produced by RSV inoculation and RSV-resistant identification.

Transgenic rice overexpressing OsHIR3 gene with basic resistance was produced by Xoo inoculation and identification.

The transgenic rice overexpressing OsHIR3 gene produced in this invention is mainly used for plants defense against RSV and Xoo infection, alleviating virus and bacterial diseases. This invention has important theoretical and practical significance for producing transgenic plants with basic resistance, and also plays pivotal role in other fields of plant disease control. Compared with the resistant strains produced by other anti-virus strategies, the main advantage of the present invention is that OsHIR3 gene is an endogenous gene in plant, and has obvious safety compared with other viral resistance genes or fragments, meanwhile transgenic rice overexpressing OsHIR3 gene gains broader basic resistance to viral and bacterial disease.

In summary, the HIR3 genes in this invention have significant differences with the traditional HIR1 genes not only in homology, but also in function. The transgenic plants overexpressing HIR3 gene elevated SA level and had basic resistance. Furthermore, the transgenic plants overexpressing HIR3 gene showed resistance to viruses by elevating SA level and inducing the up-regulated expression of receptors in SA regulatory pathway. HIR3 gene positively regulate SA pathway to elevate SA level.

Therefore, no matter what kind of plant HIR3 gene comes from, it can be obtained by cloning. The obtained HIR3 gene can be transferred into any plant by transgene method, thus providing plants with basic resistance. On one hand, this invention provides a method for manufacturing transgenic plants, including transferring exogenous HIR3 gene into plants. In some embodiments, plants are N. benthamiana or rice. In some embodiments, the method of transgene can be varied, such as transferring plasmid to Agrobacterium tumefaciens, then transferring gene to plant tissue by infecting plant tissue with Agrobacterium tumefaciens, or transferring HIR3 gene by leaf disc method or rice mature embryos derivative method. In some embodiments, the HIR3 genes were shown as SEQ ID NO:1, SEQ ID NO:2 and SEQ ID NO:23, respectively. In some embodiments, the HIR3 sequence is derived by RSV infection. In some embodiments, the obtained HIR3 genes come from N. benthamiana or rice. In some embodiments, the HIR3 genes are derived from N. benthamiana or Oryza sativa L. ssp. japonica. cv. Nipponbar.

Beneficial Effects

This invention has important theoretical and practical significance for producing transgenic plants with basic resistance, and also plays pivotal role in other fields of plant disease control. Compared with the resistant strains produced by other anti-virus strategies, the main advantage of the present invention is that HIR3 genes are endogenous genes in plants, and have obvious safety compared with other viral resistance genes or fragments, meanwhile transgenic plants overexpressing HIR3 gene gain broader basic resistance to viral and bacterial disease.

### BRIEF DESCRIPTION OF THE DRAWINGS

FIGS. 1A and 1B: Map of expression vector containing NbHIR3.1 or NbHIR3.2 gene.

FIG. 1C: Map of inserted plasmid pCV-eGFP-N1 vector.

FIGS. 2A and 2B: Molecular Biological Detection of transgenic N. benthamiana plants overexpressing NbHIR3.1 or NbHIR3.2 gene

FIGS. 3A, 3B, and 3C: Resistance analysis of transgenic N. benthamiana plants overexpressing NbHIR3.2 gene against to RSV infection.

FIGS. 4A, 4B, and 4C: Resistance analysis of transgenic N. benthamiana plants overexpressing NbHIR3.1 gene against to RSV infection.

FIGS. 5A, 5B, and 5C: Resistance analysis of transgenic N. benthamiana plants overexpressing NbHIR3.2 gene against to TuMV infection.

FIGS. 6A, 6B, and 6C: Resistance analysis of transgenic N. benthamiana plants overexpressing NbHIR3.2 gene against to PVX infection.

FIG. 7: Resistance analysis of transgenic N. benthamiana plants overexpressing NbHIR3.2 gene against to Pst. DC3000 infection.

FIGS. 8A and 8B: The regulatory mechanism of basic resistance mediated by NbHIR3. FIG. 8A shows that SA content in wild type (WT) is very low, while SA content in transgenic plants is significantly elevated. FIG. 8B shows the expression level of key genes involved in SA pathway. The expression level of these key genes (EDS1, NPR1 and PR1genes) was significantly up-regulated and SA content was elevated in three independent transgenic lines overexpressing NbHIR3 (OX7, OX10 and OX11).

FIG. 9 shows the sequence of NbHIR3.1gene (SEQ ID NO: 1).

FIG. 10 shows the sequence of NbHIR3.2gene (SEQ ID NO: 2).

FIG. 11A: Map of expression vector containing OsHIR3 gene.

FIG. 11B: Map of inserted plasmid pCV-eGFP-N1 vector.

FIGS. 12A, 12B, 12C, and 12D: Molecular Biological Detection of transgenic rice plants overexpressing OsHIR3 gene.

FIG. 13: Development phenotype of transgenic rice plants overexpressing OsHIR3 gene.

FIGS. 14A, 14B, and 14C: Resistance analysis of transgenic rice plants overexpressing OsHIR3 gene against to RSV infection.

FIGS. 15A and 15B: Resistance analysis of transgenic rice plants overexpressing OsHIR3 gene against to Xoo infection.

FIGS. 16A and 16B: The regulatory mechanism of basic resistance mediated by OsHIR3. FIG. 16A shows that SA content in three independent transgenic lines overexpressing OsHIR3 (OE6, OE8 and OE12) is significantly higher than wild type (WT). FIG. 16B shows the expression levels of key genes involved in SA pathway. The expression level of these key genes (PBZ1, PR1 and PR5 genes) was significantly up-regulated and SA content was elevated in three independent transgenic lines overexpressing OsHIR3 (OE6, OE8 and OE12).

FIG. 17: The sequence of OsHIR3 gene (SEQ ID NO:23).

### DETAILED DESCRIPTION

It should be noted that the present implementation illustrates the new functions of the genes we have discovered by several embodiments only. The validity of the present invention is verified on the model plant N. benthamiana or rice, but it should not be considered to be a limitation of the present invention.

### Embodiment 1: Cloning of NbHIR3.1 and NbHIR3.2 Gene

The NbHIR3.1 and NbHIR3.2 gene sequences were amplified by common PCR using primers HIR3.1-ORF-f and HIR3.1-ORF-r, HIR3.2-ORF-f and HIR3.2-ORF-r and N. benthamianac DNA as template, respectively. The nucleotide sequences of the NbHIR3.1 and NbHIR3.2 were shown in SEQ ID NO:1 and SEQ ID NO:2, respectively.

The cloning process is as below:

1.1 The primers used in cloning process are as follows:

Upstream primer: HIR3.1-ORF-f:
(SEQ ID NO: 3)
5′-ATGGAAATGCTAACTGTGTATTGTG-3'
Downstream primer: HIR3.1-ORF-r:
(SEQ ID NO: 4)
5′-CTATTCAGCGACCTGCACTAGCTG-3′
Upstream primer: HIR3.2-ORF-f:
(SEQ ID NO: 5)
5′-ATGGGAATGCTAATTGTGTATTCTG-3′
Downstream primer: HIR3.2-ORF-r:
(SEQ ID NO: 6)
5′-CTATTCAGTGACCTGCACTAGCTG-3′

1.2 Trizol Method for Extracting Total Plant RNA

Total RNA was extracted using TRIzol reagent (Invitrogen, Carisbad, Calif., USA) according to the manufacturer's protocol. Masks and gloves should be worn during RNA extraction to avoid RNA degradation.

1. Take moderate sample in the imported 2 mL Eppendorf (EP) tube containing steel beads, and quickly oscillate for 0.5-1 minutes (min) at 18 rps (Revolutions Per Second) on the mill after quick-frozen in liquid nitrogen. After fully grinded, add moderate Trizol (1 mL/100 mg sample to ensure fully decomposition of the sample), mix it violently, and put it on ice for 5 min. Centrifugation at 4° C., 13,000 rpm for 10 min.

2. Take the supernatant in a new 2 mL EP tube, add 1/5 volume chloroform, shock for 30 seconds (s), mix well, and stand on ice for 2-3 min. Centrifuge at 4° C., 13,000 rpm for 30 min.

3. The upper layer of EP tube is the colorless water phase containing RNA, the middle white layer is the protein phase, and the bottom layer is the chloroform phase. Take the upper water phase and transfer it into a 2 mL EP tube. Repeat step 2.

4. Absorb the upper water phase in a new 1.5 mL EP tube, add equal volume (about 600 ml) of pre-cooled isopropanol, mix it upside and down, and put it at −70° C. for 1 hour (h). Place on ice until it is fully thawed. Centrifuge at 4° C., 13,000 rpm for 30 min.

5. Discard the supernatant, add 1 mL pre-cooled 75% ethanol (prepared with RNase-free water), wash precipitate. Centrifugation at 4° C., 13,000 rpm for 5 min.

6. Repeat step 5 to clean the residual salt thoroughly.

7. Discard the supernatant, centrifuge at 4° C., 13,000 rpm for 2 min. Use a pipette gun to carefully absorb the residual liquid and dry it at room temperature until the precipitate turn into transparent from white. The precipitate is dissolved in RNase-free water.

8. RNA concentration can be determined by ultraviolet spectrophotometer, and RNA quality can also refer to the ratio of OD260/0D280 and OD260/0D230. RNA samples were kept at −80° C.

1.3 First-strand cDNA was synthesized from 1 mg of RNA using a 21-nucleotide [oligo(dT) plus two anchoring nucleotides] or gene-specific primer.

The reverse transcription system and conditions are shown as below:

| | |
|---|---|
| RNA | 2.0 μL |
| dNTP Mixture (2.5 mM each) | 5.0 μL |
| DEPC H₂O | 9.0 μL |
| Oligo (dT)18 Primer | 2.0 μL |
| 5× Reverse Transcriptase Buffer | 5.0 μL |
| Rnase inhibitor | 1.0 μL |
| AMV Reverse Transcriptase (10 U) | 1.0 μL |
| Total volume | 25.0 μL |

Firstly, four reagents (RNA, dNTP Mixture (2.5 mM each), DEPC H2O, Oligo (dT) 18 Primer) were added to the RNA enzyme free micro EP tube. They were mixed and denatured at 70° C. for 5 min and immediately placed on ice for 2 min. Then the following three reagents (5× Reverse Transcriptase Buffer, Rnase inhibitor, AMV Reverse Transcriptase (10U)) were added. After blending, the PCR reverse transcription started under conditions as below:
42° C., 2 h→72° C., 10 min

The PCR reaction system is shown as follows:

| | |
|---|---|
| 2× Taq Master Mix | 7.5 μL |
| Upstream primer (20 μmol/L) | 0.2 μL |
| Downstream primer (20 μmol/L) | 0.2 μL |
| cDNA | 1.0 μL |
| ddH₂O | 6.1 μL |
| Total volume | 15 μL |

After mixing, the PCR cycles was carried out as below:

| 94° C. | 3 min | | |
|---|---|---|---|
| 94° C. | 30 sec | | |
| 56° C. | 30 sec | {close oversize brace} | 34 cyc1es |
| 72° C. | 1 min | | |

| | |
|---|---|
| 72° C. | 10 min |

The full-length sequences of NbHIR3.1 and NbHIR3.2 were obtained from amplification, which were shown as SEQ ID NO:1 and SEQ ID NO:2, respectively.

The NbHIR3.1 and NbHIR3.2 genes in this invention were respectively 58%, 57%, 57% and 58%, 57%, 56% identical to known CaHIR1 (Accesion No. AY529867), OsHIR1 (Accesion No. NM_001068279) and AtHIR1 (Accesion No. NM_125669) genes. According to the low homology, they are two different new genes. The amino acids of NbHIR3.1 and NbHIR3.2 proteins in this invention were respectively 57%, 57%, 57% and 56%, 56%, 56% identical to those of known CaHIR1, OsHIR1 and AtHIR proteins. According to the low homology, they are two different new proteins.

### Embodiment 2: Vector Construction

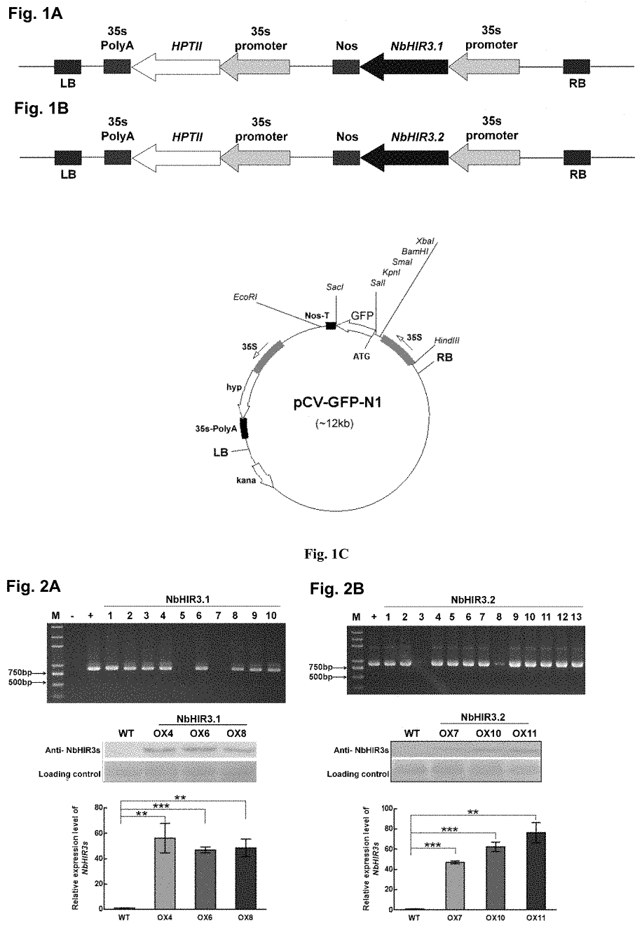

The sequences of NbHIR3.1 and NbHIR3.2 genes with corresponding restriction sites were amplified using primers HIR3.1-f and HIR3.1-r, HIR3.2-f and HIR3.2-r, and full-length NbHIR3.1 and NbHIR3.2 sequences obtained above as templates under the same PCR condition, respectively. The NbHIR3.1 and NbHIR3.2 sequences were linked to the polyclonal sites of the binary expression vector pCV1300 (Map of pCV-eGFP-N1 vector was shown in FIG. 1.2), respectively. The map of constructed vector containing NbHIR3.1 or NbHIR3.2 was shown in FIGS. 1A and 1B. More concretely, the location of GFP (fluorescent protein) is replaced by the target gene NbHIR3.1 or NbHIR3.2 to form a complete plasmid vector. Such plasmid vectors are easy to understand, and any plasmid vectors can be used, and they are also commonly used in this field.

The detection primers were shown as follows (underlined ggatcc and gagctc indicated BamHI and SalI restriction site, respectively):

HIR3.1-f:
(SEQ ID NO: 7)
5′-GggatccATGGGAAATGCTAACTGTGTATTTTGTG-3′
HIR3.1-r:
(SEQ ID NO: 8)
5′-GgagctcCTATTCAGCGACCTGTGCACTAGCTG-3′
HIR3.2-f:
(SEQ ID NO: 9)
5′-GggatccATGGGGAATGCTAATTGTGTATTCTG-3′
HIR3.2-r:
(SEQ ID NO: 10)
5′-GgagctcCTATTCAGTGACCTGTGCACTAGCTG-3′

### Embodiment 3: Agrobacterium Transformation and Positive Clones Identification

The positive plasmid was transferred into Agrobacterium tumefaciens by electric shock method. Steps are shown as follows:

(1) Add 1 μL purified plasmid DNA (Embodiment 2) into the unfrozen Agrobacterium tumefaciens stain EHA105 (unfrozen on ice), mixed gently, and then be added to the electric shock cup;

(2) Put the electric shock cup in the electric shock groove (the voltage of the electric shock meter is 2.2 kV), press the electric shock button until hearing the dripping sound.

(3) Bacterial solution was absorbed into EP tube, add 900 μL non-resistant LB medium, shaking culture at 28° C., 220 rpm for 1 hour (h).

(4) 200 μL bacterial solution were spread on LB plate culture medium (containing 50 μg/mL kanamycin (Kan) and 50 μg/mL rifampicin (Rif)) and cultured at 28° C. for 2 days (d).

Single colony of transformed Agrobacterium tumefaciens was inoculated in LB liquid medium containing 50 μg/ml Kan and 50 μg/ml Rif, shaking overnight at 28° C., 220 rpm. 1 μL bacterial solution was taken for PCR detection. The detection primers were HIR3-f and HIR3-r. The positive bacterial liquid was mixed with 30% glycerol and stored in a glycerol tube at −70° C.°C.

The detection primers were shown as below:

HIR3-f:
(SEQ ID NO: 11)
5′-AGGAGCAGATTCAGGCTTATG-3′
HIR3-r:
(SEQ ID NO: 12)
5′-CCACCTAAATACTTGGCTTCAG-3′

The PCR reaction system is shown as below:

| | |
|---|---|
| 2× Taq Master Mix | 7.5 μL |
| Upstream primer (20 μmol/L) | 0.2 μL |
| Downstream primer (20 μmol/L) | 0.2 μL |
| Bacterial solution | 1.0 μL |
| ddH₂O | 6.1 μL |
| Total volume | 15 μL |

After mixing, the PCR cycles was carried out as below:

| 94° C. | 3 min | | |
|---|---|---|---|
| 94° C. | 30 sec | | |
| 56° C. | 30 sec | {close oversize brace} | 30 cycles |
| 72° C. | 1 min | | |

| | |
|---|---|
| 72° C. | 10 min |

### Embodiment 4: Transgenic Plants Produced by Leaf Disc Method

1) Preparation of Bacterial Solution

The positive transforming strains (embodiment 3) preserved at −70° C. were streaked on the LB plate medium containing 50 μg/ml Kan and 50 μg/ml Rif at 28° C. until single colonies formation. The single colonies were selected and shaking cultured in LB solution containing 50 ug/ml Kan and 100 ug/ml Rif overnight at 28° C.,220 rpm. The bacterial solution was diluted with fresh LB solution (1:100) and then shaking cultured at 28° C., 220 rpm until OD600=1.

2) Transgenic Plants Produced by Leaf Disc Method

Transgenic N. benthamiana plants were produced by leaf disc method. The steps were shown as below:

1. Pre-culture: Leaves of N. benthamiana at on 5-6 leaf stage with good growth were selected. The leaves were washed several times with ddH2O, then sterilized with 75% ethanol for 1 min, and then been washed several times with ddH2O. The leaves were placed on aseptic filter paper to absorb the moisture of N. benthamiana leaves surface.

2. The edges and veins were removed from the sterilized leaves, then cut it into 1 cm² pieces and placed on MS pre-culture medium (containing 2 mg/L 6-BA), pre-cultured at 26° C. until the incision began to expand.

3. Agrobacterium tumefaciens containing over-expression vectors were activated on LB medium containing Kan and Rif, then transferred to liquid solution, and cultured by shaking overnight at 28° C., 220 rpm. Bacterial solution was collected by centrifuging at 4,000 rpm for 10 min.

4. Co-culture: Resuspend the collected bacteria with MS solution (including 1 μL/mL AS) and place at room temperature for 30 min. Place the pre-cultured N. benthamiana leaves in the suspension, soak at room temperature for 10 min and shake continuously to ensure fully infiltration. The leaves were placed on aseptic filter paper to absorb the surface moisture of N. benthamiana leaves surface. Leaves were cultured in co-culture medium (containing 3 mg/L 6-BA, 0.2 mg/L NAA, 100 μM AS) at 26° C. for 2-3 days.

5. Selective culture: Wash the co-cultured leaves with ddH2O several times, and then place the leaves on aseptic filter paper to absorb the moisture of the leaves surface. Leaves were cultured on MS selective medium (containing corresponding antibiotics) and been screened every two weeks.

6. Rooting culture: When the leaves grow 1 cm resistant buds on the medium, select the buds with good growth and remove the callus at the base of buds, and then transfer them to the rooting medium (containing corresponding antibiotics) for rooting culture.

7. Take the rooted seedlings, wash away the residual culture medium from the roots and transplant them into the soil (Firstly, the seedlings were cultured in the dark for 1-2 d to adapt to the external environment conditions, and then transfer them to the normal light environment for cultivation).

Ten lines of transgenic N. benthamiana plants overexpressing NbHIR3.1 and 12 lines of transgenic N. benthamiana plants overexpressing NbHIR3.2 were produced by leaf disc method.

3) Molecular Biological Detection of Transgenic Plants

CTAB method was used to extract the DNA of transgenic N. benthamiana plants overexpressing NbHIR3.1 or NbHIR3.2. The steps were shown as below:

\textcircled{1} Put proper amount of plant material into 2 mL EP tube, grind it completely by liquid nitrogen, add 500 μL 2×CTAB, and shock violently.

\textcircled{2} EP tubes were bathed at 65° C. for 30 min, mixed upside and down every 10 min.

\textcircled{3} Add 500 mL chloroform, shake to mix, centrifuge at room temperature at 12,000 rpm for 10 min, and take the supernatant into the new EP tubes.

\textcircled{4} Repeat step \textcircled{3} once.

\textcircled{5} Add equal volume of isopropanol and 1/10 volume of NaOAc (3M, pH 5.2), mix well, and store at −20° C. for 15 min.

\textcircled{6} Centrifuge at room temperature at 12,000 rpm for 10 min.

\textcircled{7} The supernatant was discarded and the precipitation was washed with 75% ethanol and centrifuged at room temperature at 12,000 rpm for 5 min.

\textcircled{8} Repeat step \textcircled{7} once.

\textcircled{9} Discard the supernatant, open the cap and place it at room temperature for 15 min to dry the precipitation. The precipitation is dissolved in 40 μL ddH2O.

Since NbHIR3s are endogenous genes of N. benthamiana, specific primers (HIR3-f: 5′-AGGAGCAGATTCAGGGCTTATG-3′ (SEQ ID NO: 13)) and vector primers (NOS-r: 5′-GATAATCATCGCAAGACCGG-3′ (SEQ ID NO: 14)) were selected for PCR detection. The results showed that 8 lines of transgenic N. benthamiana plants overexpressing NbHIR3.1 and 11 lines of transgenic N. benthamiana plants overexpressing NbHIR3.2 were positive (FIG. 2A and FIG. 2B), and the positive rates were 80% and 85%, respectively.

Total RNA and protein were extracted respectively from leaves of positive transgenic lines and detected by qRT-PCR and Western blot. The results showed that the expression levels of HIR3s in these positive lines were different. The mRNA and protein levels of HIR3s in three independent transgenic lines overexpressing NbHIR3.2(lines OX7, OX10 and OX11) and three independent transgenic lines overexpressing NbHIR3.1 (lines OX4, OX6 and OX8) were significantly higher than those in wild-type (WT) plants (FIG. 2, WT indicate wildtype, others indicate positive lines).

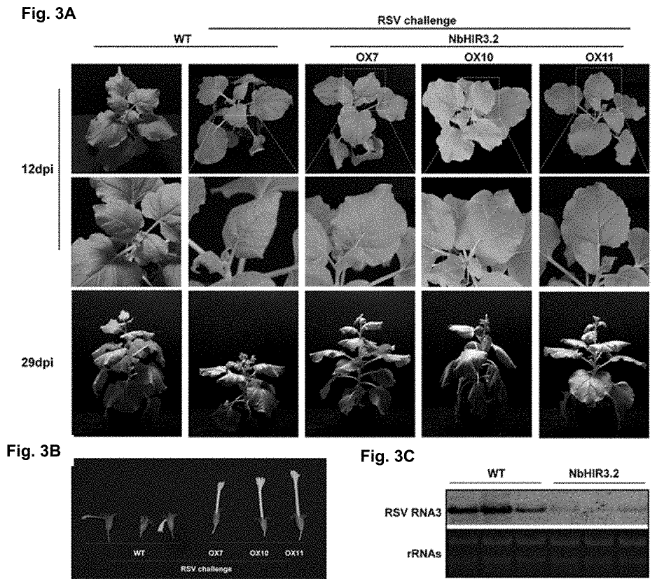

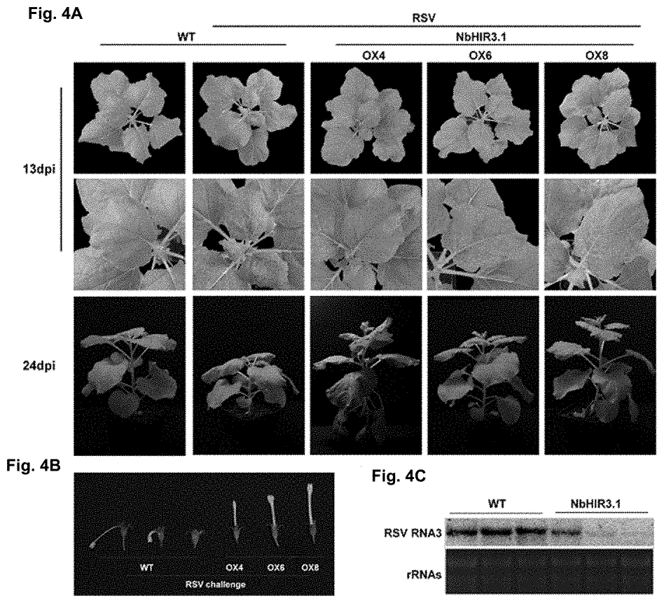

Phenotypic observation showed that All positive transgenic lines developed normally without obviously changed phenotype compared with WT, indicated that overexpression of NbHIR3s did not affect the normal growth and development of N. benthamiana (FIGS. 3A-C, FIGS. 4A-C).

### Embodiment 5: Analysis of Transgenic N. benthamiana Plants Against to RSV

This invention selected T2 generation of transgenic N. benthamiana plants overexpressing NbHIR3.1 or NbHIR3.2 for RSV inoculation identification.

The steps were shown as below:

1. N. benthamiana are Inoculated with RSV by Rubbing Leaves

N. benthamiana at 5-6 leaf stage was inoculated with RSV by rubbing leaves and the wild type N. benthamiana with the same growth was used as control. Two opposite leaves at the same leaf position of each N. benthamiana plants were selected for inoculation. The RSV-infected rice leaves were cut into small segments and ground into powder by liquid nitrogen, and then they were transferred to inoculation buffer (0.1M PBS, pH 7.0) and then ground to liquid state. The same amount of liquid with RSV was taken to each leaf for RSV inoculation. The N. benthamiana plants with the same growth were inoculated with inoculation buffer as control. The N. benthamiana plants were transferred to the normal greenhouse environment for cultivation under the same environment after inoculation.

2. RSV Inoculation Identification of Transgenic N. benthamiana Plants Overexpressing NbHIR3.1 or NbHIR3.2

After inoculation with RSV, all wild-type plants showed leaf-twisting, chlorotic, yellow-green stripes which were the typical symptoms of systemic infection with RSV at 12 dpi. In contrast, only about 70% plants of lines overexpressing NbHIR3.2 showed mild symptoms while the remainder were symptom-free (FIG. 3A). At 29 dpi, the statistics of plant height showed that the plant height of N. benthamiana overexpressing NbHIR3.2 was significant higher than control (FIG. 3A). Meanwhile, all wild-type plants had typical severe symptoms, such as stunting and deficiency in flower development, while plants overexpressing NbHIR3.2 had only very mild symptoms or none at all (FIG. 3B).

Northern blot analysis of the RSV RNA levels showed that plants from three different lines OX7, OX10 and OX11 had significant lower levels of RSV RNAs than wild-type plants (FIG. 3C). Similar results were obtained using three lines of plants overexpressing NbHIR3.1 (FIGS. 4A-C). These results demonstrate that overexpression of NbHIR3s reduced accumulation of RSV RNAs in N. benthamiana, and obtained the tolerance to RSV infection.

### Embodiment 6: Analysis of Transgenic N. benthamiana Plants Against to Turnip Mosaic Virus

The embodiment 1 indicated that N. benthamiana overexpressing NbHIR3.1 or NbHIR3.2 showed the tolerance to RSV. In order to clarify that NbHIR3s-mediated resistance also targets to other viruses, the transgenic N. benthamiana plants overexpressing NbHIR3.1 or NbHIR3.2 were inoculated with Turnip Mosaic Virus (TuMV, typical of Potato Y Virus) and Potato VirusX (PVX, typical of Potato X Virus), respectively.

1. Analysis of NbHIR3.2 Transgenic N. benthamiana Plants Against to Turnip Mosaic Virus (TuMV)

N. benthamiana at 5-6 leaf stage was inoculated with TuMV-GFP and the wild type N. benthamiana with the same growth was used as control. Two opposite leaves at the same leaf position of each N. benthamiana plants were selected for inoculation. The TuMV-infected N. benthamiana leaves were transferred to inoculation buffer (0.1M PBS, pH 7.0), and then ground to liquid state. The same amount of liquid with RSV was taken to each leaf for TuMV inoculation. The N. benthamiana plants were transferred to the normal greenhouse environment for cultivation under the same environment after inoculation.

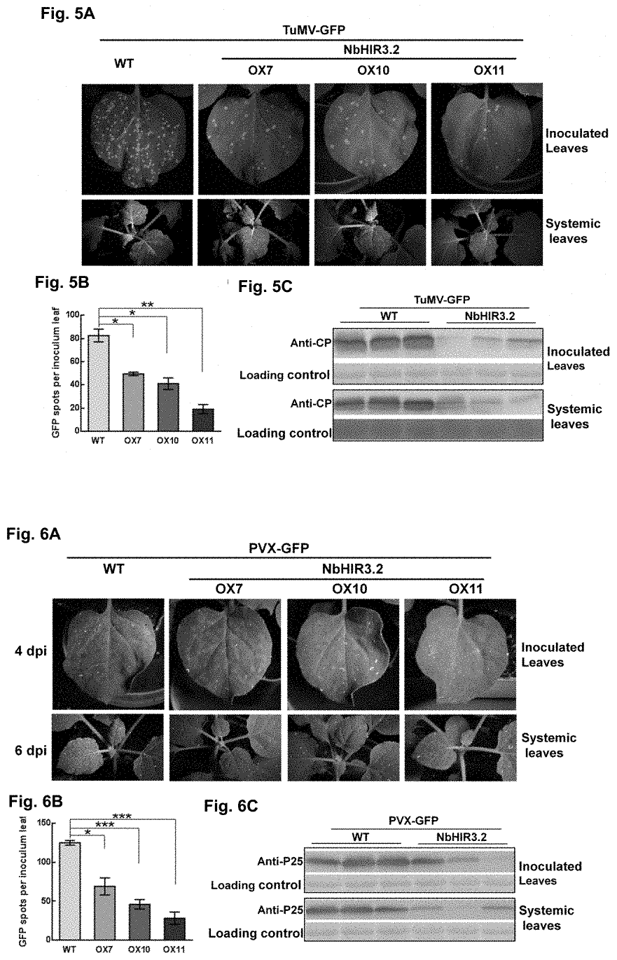

Continuous monitoring of virus development showed that 60 h after inoculated with infectious clone TuMV-GFP, GFP fluorescent spots appeared on the inoculated leaves of N. benthamiana (FIG. 5A). The statistical analysis showed that the number of fluorescent spots on the inoculated leaves of transgenic N. benthamiana plants overexpressing NbHIR3.2 significantly decreased compared with the control (FIG. 5A, 5B). 4.5 d after inoculation, typical TuMV symptoms (e.g. curling, wavy and chimerism) and strong GFP fluorescence appeared in systemic leaves of wild-type N. benthamiana, while transgenic N. benthamiana plants overexpressing NbHIR3.2 showed slight symptoms and scattered GFP fluorescence (FIG. 5A).

Western blot showed that the expression level of TuMV-CP in the inoculated leaves and systemic leaves of transgenic N. benthamiana plants overexpressing NbHIR3.2 was significant lower than control (FIG. 5C). These results indicated that overexpression of NbHIR3s confer plants resistance to TuMV.

Similar results were obtained using three lines of plants overexpressing NbHIR3.1 after inoculated with TuMV.

2. Analysis of NbHIR3.2 Transgenic N. benthamiana Plants Against to Potato Virus X(PVX)

N. benthamiana at 5-6 leaf stage was inoculated with PVX-GFP and the wild type N. benthamiana with the same growth was used as control. Two opposite leaves at the same leaf position of each N. benthamiana plants were selected for inoculation. The PVX-infected N. benthamiana leaves were transferred to inoculation buffer (0.1M PBS, pH 7.0), and then ground to liquid state. The same amount of liquid with RSV was taken to each leaf for PVX inoculation. The N. benthamiana plants were transferred to the normal greenhouse environment for cultivation under the same environment after inoculation.

Continuous monitoring of virus development showed that 4 days after inoculated with infectious clone PVX-GFP, GFP fluorescent spots appeared on the inoculated leaves of N. benthamiana (FIG. 6A). The statistical analysis showed that the number of fluorescent spots on the inoculated leaves of transgenic N. benthamiana plants overexpressing NbHIR3.2 significantly decreased compared with the control (FIG. 6A, 5B). 6 days after inoculation, typical PVX symptoms and dense distribution of GFP fluorescence appeared in systemic leaves of wild-type N. benthamiana, while the systemic leaves of transgenic N. benthamiana plants overexpressing NbHIR3.2 showed slight symptoms and scattered GFP fluorescence (FIG. 6A).

Western blot showed that the expression level of PVX-P25 in the inoculated leaves and systemic leaves of transgenic N. benthamiana plants overexpressing NbHIR3.2 was significant lower than control (FIG. 6C). These results indicated that overexpression of NbHIR3s confer plants resistance to PVX.

Similar results were obtained using three lines of plants overexpressing NbHIR3.1 after inoculated with PVX.

### Embodiment 7: Analysis of Transgenic N. benthamiana Plants Against to Pst. DC3000

The embodiment 5, 6 indicated that N. benthamiana overexpressing NbHIR3.1 or NbHIR3.2 showed the tolerance to RSV. Previous studies showed that transgenic Arabidopsis plants overexpressing HIR1 gene exhibit resistance to Pst. DC3000. In order to clarify that NbHIR3s-mediated resistance also targets to other pathogens, the transgenic N. benthamiana plants overexpressing NbHIR3.2 were inoculated with Pst. DC3000.

1) Inoculation with Pst. DC3000

N. benthamiana at 5-6 leaf stage was inoculated with Pst. DC3000 and the wild type N. benthamiana with the same growth was used as control. Fresh Pst. DC3000 colonies which activated twice were selected with toothpicks and diluted to OD600=0.0002 with 10 mM MgCl2. The selected bacteria buffer was injected into the leaves with a 1 mL needle-free syringe from the back of the leaves. The N. benthamiana was transferred to the normal greenhouse environment for cultivation under the same environment after inoculation.

2) Determination of CFU Value of Pathogenic Bacteria

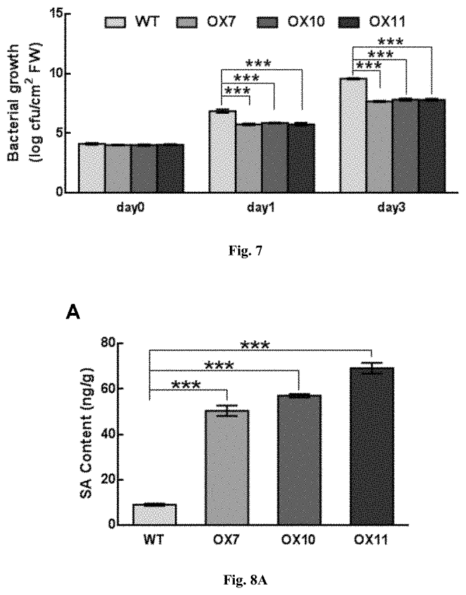

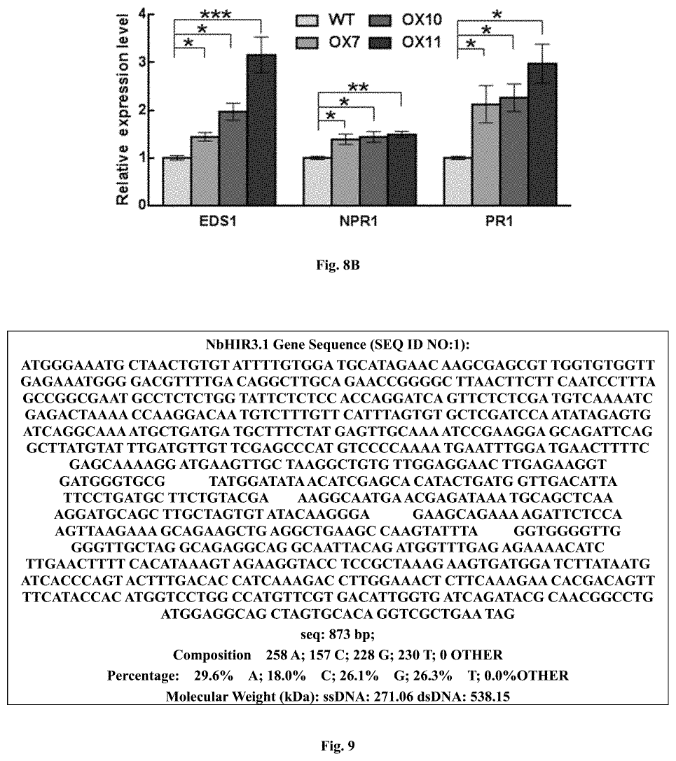

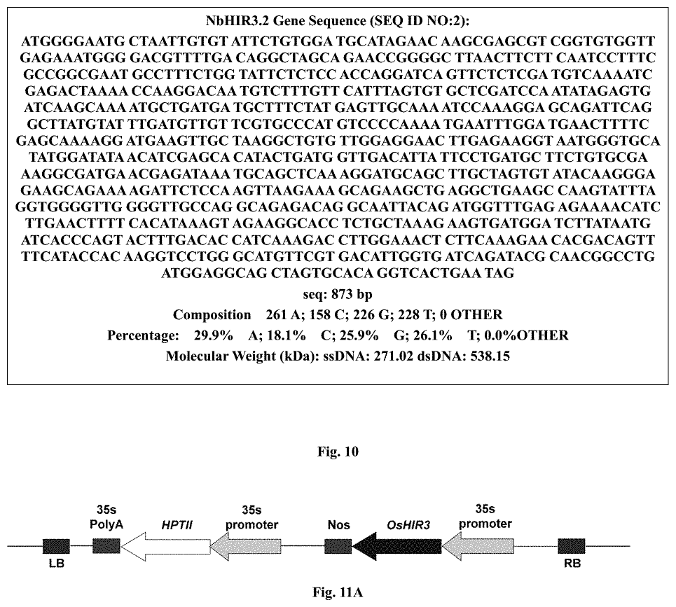

Continuous symptoms observation was taken 0-3 days after inoculation. Leaves with symptoms of bacterial infection were cut off and soaked in ddH2O for 1 min and then washed by ddH2O 2-3 times. Leaf discs of the same size were obtained with a 0.5 cm diameter perforator and grinded to homogenate with 100 μL ddH2O, then washed the grinding rod with 900 μL ddH2O and mixed it with homogenate. Gradient dilution was carried out after mixing. The 10-ml diluent was applied to KB plate medium containing 50 μg/mL Kan and 50 μg/mL Rif. The number of colonies was counted and the CFU value of pathogenic bacteria was calculated after incubating in constant-temperature incubator for 2 days. Each experiment was repeated three times. The results showed that 3 days after inoculation with Pst. DC3000, The accumulation of Pst. DC3000 was significantly reduced in NbHIR3.2 transgenic N. benthamiana plants compared with wild-type (FIG. 7), indicating that NbHIR3.2 confers plants resistance to Pst. DC3000.

In conclusion, NbHIR3s-mediated basic resistance not only targets to RSV, but also other pathogens, such as TuMV, PVX and Pst. DC3000. This indicates that NbHIR3.1 and NbHIR3.2 confer plants basic resistance to virus and bacteria. Compared with the traditional HIR1, NbHIR3.1 and NbHIR3.2 genes gain broader basal resistance and have wider application prospects.

### Embodiment 8: NbHIR3s Confer Plants Basic Resistance by Positively Regulating SA Pathway

The positive lines with high expression level of NbHIR3s (NbHIR3.1 and NbHIR3.2) and basal resistance were selected through the above tests. The SA content and the qRT-PCR analysis of key genes involved in SA pathway were detected in those positive lines. Referring to the qRT-PCR instructions, the steps are shown as below:

| | |
|---|---|
| SYBR Green Realtime PCR Master Mix | 18.0 μL |
| cDNA | 6.0 μL |
| Upstream primer | 3.6 μL |
| Downstream primer | 3.6 μL |
| RNase-free H2O | 10.8 μL |
| Total volume | 36.0 μL |

The reagents were added to RNase-free EP tube in turn, fully mixed, centrifuged instantaneously, added into 384 holes quantitative plate with 10 μL/hole, coated with membrane, and placed in qRT-PCR machine to react at 95° C. for 5 min, 40 cycles: 95° Cfor 20 s→58° C. for 20 s→72° C. for 20 s, 72° C. for 10 min.

The specific primers used for qRT-PCR analysis are shown as below:

| Primer name | Sequence |
|---|---|
| RT-Actin-f | 5′-AAGACCAGCTCATCCGTGGA-3′ |
| | (SEQ ID NO: 15) |
| RT-Actin-r | 5′-CTCATCCTATCAGCAATGCCC-3′ |
| | (SEQ ID NO: 16) |
| RT-NbEDS1-f | 5′-TGGAAATGGGAAACTGGTGGTC-3′ |
| | (SEQ ID NO: 17) |
| RT-NbEDS1-r | 5′-GACAAGGGAATATCGGTAAGATTATTG-3′ |
| | (SEQ ID NO: 18) |
| RT-NbNPR1-f | 5′-GAAACGCCTATCGGAAACACTG-3′ |
| | (SEQ ID NO: 19) |
| RT-NbNPR1-r | 5′-AAGCCAATACACTCATTACAGCATC-3′ |
| | (SEQ ID NO: 20) |
| RT-NbPR1-f | 5′-ACAAGACTATTTGGATGCCC-3′ |
| | (SEQ ID NO: 21) |
| RT-NbPR1-r | 5′-TCTCAACAGCCTTAGCAGC-3′ |
| | (SEQ ID NO: 22) |

There was significant more SA in the three lines overexpressing NbHIR3.2 than in the wild-type plants (FIG. 8A) and the expression levels of EDS1, NPR1 and PRlwhich were key genes involved in SA pathway were also upregulated ((FIG. 8B). This indicated that NbHIR3s confer plants basic resistance by positively regulating SA pathway.

This is because after plants infected by pathogens, systemic resistance causes the uninfected parts at the distal end to generate resistance to pathogens, which is called systemic acquired resistance (SAR), and this phenomenon has been confirmed in many models of interactions between plants and pathogens. The typical characteristics of SAR is restricting the growth of pathogens and inhibiting the development of infection symptoms. The role of SA in SAR has been reported many times in plants. The mainstream view is that SA is an important signal molecule in the SAR process, and the accumulation of SA will stimulate SAR response. The high expression level of pathogenesis related protein (PR protein) is an important symbol of SAR reaction. And multiple PR proteins always coordinate together instead of a specific PR protein acting alone to cause SAR reaction. PR protein accumulated in tobacco treated with SA or aspirin and confer resistance to Tobacco mosaic virus (TMV) infection. TMV infection can induce a sharp increase of endogenous SA content of tobacco, and the SA content of resistant varieties is significantly higher than that of susceptible varieties. Neither SA accumulating nor SAR activating will happen in the sid1 and sid2 mutant plants and the sidl and sid2 mutant plants showing sensitivity to Pseudomonas syringae. Those reports further proved that SA is a key signal molecule in SAR process.

Regulatory protein NPR1 is a key component in SA-mediated signal transduction pathway. NPR1 can induce the expression of PR-1 and other resistant genes, thus enhancing the disease resistance of plants. During pathogens infection, SA level is normal, but SAR cannot be induced in niml mutant plants, in which the expression of NPR1 gene is affected, indicating that NPR1 acts on downstream of SA and is a key regulatory factor in SAR signal transduction pathway. Despres et.al found that NPR1 can interact with members of Arabidopsis thaliana TGA family which rich in leucine (bZIP) transcription factors, while NPR1 mutant loses its interaction with TGA2, indicating that NPR1-mediated TGA2 binding is critical to the activation of defense genes. When SAR is induced, NPR1 activated PR-1 gene through interaction with transcription factors in the promoter region of PR gene, indicating that the activity of NPR1 is closely related to the regulation of the expression of PR genes. The four-point mutant of NPR1 blocked SA signal and lost interaction with TGA2 and TGA3. TGA2 and TGA3 are able to bind SA response elements of Arabidopsis thaliana PR-1 promoter, and NPR1 and SA-induced PR-1 gene expression were linked by the TGA transcription factor.

EDS1 and NDR1 are two independent positive regulators locating upstream in SA pathway, since the two proteins locate downstream of two functionally distinct classes of R proteins. EDS1 is a positive regulator of basal resistance to pathogen invasion and R protein-mediated resistance. And EDS1 is also indispensable for Toll-Interleukin-1 receptor (TIR)-type nucleotide binding-leucine rich repeat (NB-LRR) protein-triggered resistance. EDS1 can interacted with PAD4 AND SAG101, forming a complex in the cytoplasm and nucleus and inducing SA accumulation. SA can also induce the expression of R, EDS1, PAD4 and SID2 genes through feedback mechanism, boosting SA signal. NDR1 is another SA positive regulator acting independently from EDS1. NDR1 is required for resistance induced by many R genes encoding CC-NBS-LRR proteins.

### Embodiment 9: Cloning of OsHIR3 Gene

Plants used in this invention are Oryza sativa L. ssp. japonica. cv. Nipponbare.

1. Acquisition of recombinant Agrobacterium tumefaciens

1) Cloning of OsHIR3 Gene

The OsHIR3 gene sequence was amplified by common PCR using primers OsHIR3-ORF-f and OsHIR3-ORF-r and Oryza sativa L. ssp. japonica. cv. Nipponbare cDNA as template. The nucleotide sequences of the OsHIR3gene was shown in SEQ ID NO:23.

The cloning primers are shown as below:

OsHIR3-ORF-f:
(SEQ ID NO: 24)
5′-ATGGTGAGCGCCTTCTTCCTGCT-3′
OsHIR3-ORF-r:
(SEQ ID NO: 25)
5′-TTACACGTTGCTGCAGGACGCTT-3′

Total RNA was extracted using TRIzol reagent (Invitrogen, Carisbad, Calif., USA) according to the manufacturer's protocol. Masks and gloves should be worn during RNA extraction to avoid RNA degradation.

1. Take moderate sample in the imported 2 mL Eppendorf (EP) tube containing steel beads, and quickly oscillate for 0.5-1 minutes (min) at 18 rps (Revolutions Per Second) on the mill after quick-frozen in liquid nitrogen. After fully grinded, add moderate Trizol (1 mL/100 mg sample to ensure fully decomposition of the sample), mix it violently, and put it on ice for 5 min. Centrifugation at 4° C., 13,000 rpm for 10 min.

2. Take the supernatant in a new 2 mL EP tube, add ⅕ volume chloroform, shock for 30 seconds (s), mix well, and stand on ice for 2-3 min. Centrifuge at 4° C., 13,000 rpm for 30 min.

3. The upper layer of EP tube is the colorless water phase containing RNA, the middle white layer is the protein phase, and the bottom layer is the chloroform phase. Take the upper water phase and transfer it into a 2 mL EP tube. Repeat step 2.

4. Absorb the upper water phase in a new 1.5 mL EP tube, add equal volume (about 600 ml) of pre-cooled isopropanol, mix it upside and down, and put it at −70° C. for 1 hour (h). Place on ice until it is fully thawed. Centrifuge at 4° C., 13,000 rpm for 30 min.

5. Discard the supernatant, add 1 mL pre-cooled 75% ethanol (prepared by with RNase-free water), wash precipitate. Centrifugation at 4° C., 13,000 rpm for 5 min.

6. Repeat step 5 to clean the residual salt thoroughly.

7. Discard the supernatant, centrifuge at 4° C., 13,000 rpm for 2 min. Use a pipette gun to carefully absorb the residual liquid and dry it at room temperature until the precipitate turn into transparent from white. The precipitate is dissolved in RNase-free water.

8. RNA concentration can be determined by ultraviolet spectrophotometer, and RNA quality can also refer to the ratio of OD260/OD280 and OD260/OD230. RNA samples were kept at −80° C.

First-strand cDNA was synthesized from 1 mg of RNA using a 21-nucleotide [oligo(dT) plus two anchoring nucleotides] or gene-specific primer.

The reverse transcription system and conditions are shown as below:

| | |
|---|---|
| RNA | 2.0 μL |
| dNTP Mixture (2.5 mM each) | 5.0 μL |
| DEPC H2O | 9.0 μL |
| Oligo (dT) 18 Primer | 2.0 μL |
| 5× Reverse Transcriptase Buffer | 5.0 μL |
| Rnase inhibitor | 1.0 μL |
| AMV Reverse Transcriptase (10 U) | 1.0 μL |
| Total volume | 25.0 μL |

Firstly, four reagents (RNA, dNTP Mixture (2.5 mM each), DEPC H2O, Oligo (dT) 18 Primer) were added to the RNA enzyme free micro EP tube. They were mixed and denatured at 70° C. for 5 min and immediately placed on ice for 2 min. Then the following three reagents (5× Reverse Transcriptase Buffer, Rnase inhibitor, AMV Reverse Transcriptase (10 U)) were added. After blending, the PCR reverse transcription started under conditions is as below:

42° C., 2 h→72° C., 10 min

The PCR reaction system is shown as follows:

| | | |
|---|---|---|
| 10× Ex Taq Buffer | 5.0 | μL |
| dNTPs (2.5 mmol/L ) | 5.0 | μL |
| Upstream primer (10 μM) | 1.0 | μL |
| Downstream primer (10 μM) | 1.0 | μL |
| Ex TaqDNA Polymerase | 0.5. | μL |
| cDNA | 0.5. | μL |
| ddH2O | 37.0 | μL |
| Total volume | 50.0 | μL |

After mixing, the PCR cycles was carried out as below:

| 94° C. | 3 min | | |
|---|---|---|---|
| 94° C. | 30 sec | | |
| 56° C. | 30 sec | {close oversize brace} | 34 cyc1es |
| 72° C. | 1 min | | |

| | |
|---|---|
| 72° C. | 10 min |

The OsHIR3 gene in this invention was 60%, 60% and 59% identical to known CaHIR1 (Accesion No. AY529867), OsHIRl (Accesion No. NM_001068279) and AtHIRl (Accesion No. NM_125669) genes. According to the low homology, it is a different new gene. The amino acids of OsHIR3 protein in this invention was 58%, 59% and 59% identical to those of known CaHIR1, OsHIR1 and AtHIR proteins. According to the low homology, it is a different new protein. The OsHIR3 gene was 80% and 81% identical to NbHIR3.1 and NbHIR3.2 genes shown above, while the amino acids of OsHIR3 protein were 74% and 73% identical to those of NbHIR3.1 and NbHIR3.2 proteins, indicating that HIR3 genes are conversed in different plants.

### Embodiment 10: Vector Construction

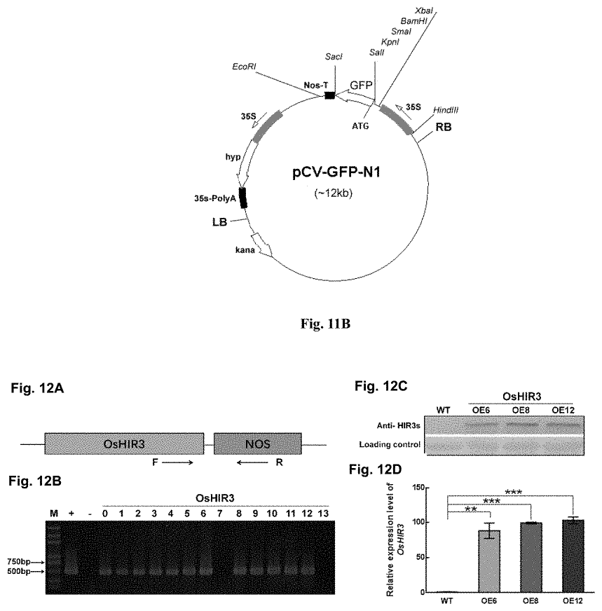

The sequences of OsHIR3 gene with corresponding restriction sites were amplified using primers OsHIR3-f and OsHIR3-r, and full-length OsHIR3 sequence obtained above as templates under the same PCR condition, respectively. The OsHIR3 sequence was linked to the polyclonal sites of the binary expression vector pCV1300 (Map of pCV-eGFP-N1 vector was shown in FIG. 11B). The map of constructed vector containing OsHIR3 was shown in FIG. 11A. More concretely, the location of GFP (fluorescent protein) is replaced by the target gene OsHIR3 to form a complete plasmid vector. Such plasmid vectors are easy to understand, and any plasmid vectors can be used, and they are also commonly used in this field.

The primers used were shown as follows (underlined TCTAGA and GGATCC indicated Xba I and BamHI restriction site, respectively):

OsHIR3-f:
(SEQ ID NO: 26)
5′-tgcTCTAGAATGGTGAGCGCCTTCTTCCTGCT-3′
OsHIR3-r:
(SEQ ID NO: 27)
5′-cgcGGATCCTTACACGTTGCTGCAGGACGCTT-3

### Example 11: Agrobacterium Transformation and Positive Clones Identification

The positive plasmid was transferred into Agrobacterium tumefaciens by electric shock method. Steps are shown as follows:

(1) Add 1 μL purified plasmid DNA (Embodiment 10) into the unfrozen Agrobacterium tumefaciens stain EHA105 (unfrozen on ice), mixed gently, and then be added to the electric shock cup;

(2) Put the electric shock cup in the electric shock groove (the voltage of the electric shock meter is 2.2 kV), press the electric shock button until hearing the dripping sound.

(3) Bacterial solution was absorbed into EP tube, add 900 μL non-resistant LB medium, shaking culture at 28° C., 220 rpm for 1 hour (h).

(4) 200 μL bacterial solution were spread on LB plate culture medium (containing 50 μg/mL kanamycin (Kan) and 50 μg/mL rifampicin (Rif)) and cultured at 28° C. for 2 days (d).

Single colony of transformed Agrobacterium tumefaciens was inoculated in LB liquid medium containing 50 μg/ml Kana and 50 μg/ml Rif, shaking overnight at 28° C., 220 rpm. 1 μL bacterial solution was taken for PCR detection. The detection primers were OsHIR3-detec-f and NOS-r. The positive bacterial liquid was mixed with 30% glycerol and stored in a glycerol tube at −70° C.

The detection primers were shown as below:

OsHIR3-detec-f:
(SEQ ID NO: 28)
5′-AAGGTGATGGGAGATTATGGTTAC-3′
NOS-r:
(SEQ ID NO: 29)
5′-GATAATCATCGCAAGACCGG-3′

The PCR reaction system is shown as below:

| | |
|---|---|
| 2× Taq Master Mix | 7.5 μL |
| Upstream primer (20 μmol/L) | 0.2 μL |
| Downstream primer (20 μmol/L) | 0.2 μL |
| Bacterial solution | 1.0 μL |
| ddH₂O | 6.1 μL |
| Total volume | 15 μL |

After mixing, the PCR cycles was carried out as below:

| 94° C. | 3 min | | |
|---|---|---|---|
| 94° C. | 30 sec | | |
| 56° C. | 30 sec | {close oversize brace} | 30 cycles |
| 72° C. | 1 min | | |

| | |
|---|---|
| 72° C. | 10 min |

### Embodiment 12: Transgenic Plants Produced by Inducing Callus from Rice Mature Embryos

1) Preparation of Bacterial Solution

The positive transforming strains (embodiment 11) preserved at −70° C. were streaked on the LB plate medium containing 50 μg/ml Kan and 50 μg/ml Rif at 28° C. until single colonies formation. The single colonies were selected and shaking cultured in LB solution containing 50 ug/ml Kan and 100 ug/ml Rif overnight at 28° C., 220 rpm. The bacterial solution was diluted with fresh LB solution (1:100) and then shaking cultured at 28° C., 220 rpm until OD600=1.

2) Transgenic plants were produced by callus induced from rice mature embryos, and the steps were shown as follows:

1. Sterilization:

\textcircled{1} The young spikes of Oryza sativa at grain filling stage were manually or mechanically threshed and hulled, and full, smooth and sterile seeds were then selected, washed by sterile water.

\textcircled{2} Put the seeds into a sterile glass tube, washed seeds with sterile water for 2-3 times.

\textcircled{3} Seeds were sterilized by 70% alcohol for 1 min, then washed with sterile water for 2-3 times.

\textcircled{4} Add 30% sodium hypochlorite (NaClO, available chlorine 5.2%, containing several drops of Tween -20) solution. Seeds were stand and soaked for 30 min and then washed with sterile water for 2-3 times, finally soaked with sterile water for 30-45 min.

2. Induction Culture:

Spread seeds on sterile filter paper to absorb excess water, then place 5-10 seeds per dish into mature embryo induction culture medium. The culture dish was sealed with sealing film and cultured in light incubator at 28° C. for about 20 days.

3. Subculture:

When the seeds grow pale yellow and compact globular embryogenic callus, the culture dish is opened in an ultra-clean workbench, and the naturally divided complete embryogenic callus is picked out by tweezers, then placed in a subculture medium, and subcultured for 1 week in light incubator 28° C. (if not used immediately, the culture dish can be moved to the dark place, and the culture can be continued for 1 week at 22° C.).

4. Co-Culture:

\textcircled{1}Agrobacterium Monoclonal was selected to shaking culture until the bacterial solution OD600 is about 1.0, Collecting bacterial solution and resuspend the solution with AAM (containing 200 μM As), adjusting OD600 to about 0.1.

\textcircled{2} Selecting callus with appropriate size, place them into the prepared Agrobacterium suspension mentioned above, and fully soaking for 5 min. Taking out the callus and dry it on sterile filter paper for 0.5-1 h. The callus was placed on the co-culture medium and cultured at 25° C. in the dark for 2-2.5 d.

5. Screening Culture:

\textcircled{1} The callus was taken out and washed with sterile water, during which it kept oscillating. The callus was washed and soaked in sterile water with 500 mg/L cefradine for 30 min, and then washed for 3 time, after that, place the callus on sterile filter paper to dry water for 2 h.

\textcircled{2} The dried callus was transferred to a selecting medium (containing 500 mg/L cefradine and 50 mg/L hygromycin) for the first round of screening, and cultured in light incubator at 28° C. for 14 days.

\textcircled{3} Select the initial callus of newly-born resistant callus and place the initial callus on a new selecting medium (containing 500 mg/L cefradine and 50 mg/L hygromycin) for a second selection, culture for about 10 days in light incubator at 28° C. until granular resistant callus grows.

6. Differentiation Culture:

Select resistant callus with bright yellow color, and transfer them to plastic jar containing differentiation culture medium (4-5 pieces in each jar), culture in constant temperature incubator, and differentiate into seedlings for 15-30 days.

7. Rooting, Strengthening Seedling and Transplanting:

When the seedlings differentiated from the callus grow to 2-3 cm height, the seedlings with root callus removed are taken out, and transferred to rooting culture medium and cultured for 1-2 weeks. Add a proper amount sterile water to the seedlings with good growth (when the seedlings grow to the top of the tube, open the cover in time), and refine the seedlings for 3-7 days. Wash off the root culture medium and transplant the seedlings to the soil. The seedlings should not be submerged by water surface and cultured in normal greenhouse environment.

14 lines of transgenic Oryza sativa plants overexpressing OsHIR3 gene were produced by callus induction from rice mature embryos.

3) Molecular Biological Detection of Transgenic Plants

CTAB method was used to extract the DNA of transgenic Oryza sativa plants overexpressing OsHIR3. The steps were shown as below:

\textcircled{1} Put proper amount of plant material into 2 mL EP tube, grind it completely by liquid nitrogen, add 500 μL 2×CTAB, and shock violently.

\textcircled{2} EP tubes were bathed at 65° C. for 30 min, mixed upside and down every 10 min.

\textcircled{3} Add 500 mL chloroform, shake to mix, centrifuge at room temperature at 12,000 rpm for 10 min, and take the supernatant into the new EP tubes.

\textcircled{4} Repeat step \textcircled{3} once.

\textcircled{4} Add equal volume of isopropanol and 1/10 volume of NaAc (3M, pH 5.2), mix well, and store at −20° C. for 15 min.

\textcircled{6} Centrifuge at room temperature at 12,000 rpm for 10 min.

\textcircled{7} The supernatant was discarded and the precipitation was washed with 75% ethanol and centrifuged at room temperature at 12,000 rpm for 5 min.

\textcircled{8} Repeat step \textcircled{7} once.

\textcircled{9} Discard the supernatant, open the cap and place it at room temperature for 15 min to dry the precipitation. The precipitation is dissolved in 40 μL ddH2O.

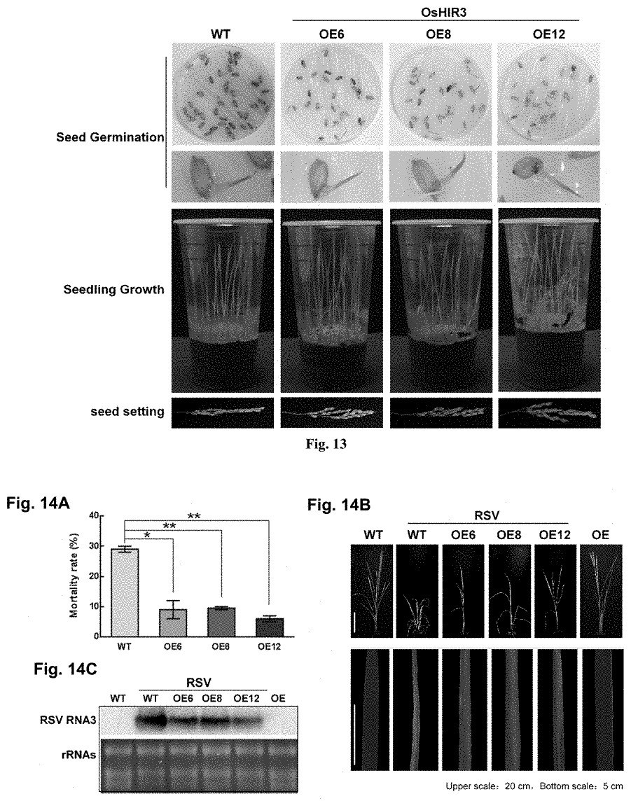

Since OsHIR3 is an endogenous gene of Oryza sativa, specific primers (OsHIR3-detec-f: 5′-AAGGTGATGGGAGATTATGGTTAC-3′ (SEQ ID NO:30)) and vector primers (NOS-r: 5′-GATAATCATCGCAAGACCGG-3′ (SEQ ID NO: 31)) were selected for PCR detection. The results showed that 12 lines of transgenic Oryza sativa plants overexpressing OsHIR3 were positive (FIG. 12B), and the positive rates were 86%.

In order to detect whether OsHIR3 gene has been integration into the genome successfully and has high expression level, total RNA and protein were extracted from leaves of positive transgenic lines and detected by qRT-PCR and Western blot. The expression levels of OsHIR3 mRNA and OsHIR3 protein in three independent transgenic lines overexpressing OsHIR3 (lines OE6, OE8 and OE12) were significantly higher than those in wild-type (WT) plants (FIG. 12C and FIG. 12D). Phenotypic observation showed that there was no significant difference in seed germination, plant seedling growth and seed setting between T2 generation transgenic rice plants and wild type (FIG. 13), indicating that the overexpression of OsHIR3 had no significant effect on growth and development of Oryza sativa.

### Embodiment 13: Analysis of Transgenic Oryza sativa Plants Against to RSV

In this invention, T2 generation of three independent transgenic lines overexpressing OsHIR3(lines OE6, OE8 and OE12) are selected for RSV inoculation identification. The steps are shown as follows:

1) Purification and Identification of Small Brown Planthopper (SBPH, LaodelphaxstriatellusFallén) with High Virus-Carrying Rate.

The SBPH population was fed with rice plants infected with RSV for 5-7 days to ensure that the SBPH could be fully poisoned. Then the 5th instar female worm was captured separately and put in a test tube (2-3 rice seedlings were planted in the test tube for feeding) for single worm feeding. The single worm was raised for 2-3 weeks (after the second generation larvae grow to 2-3 instars), 3 young SBPH were captured per tube for single worm virus-carrying rate detection by RT-PCR, and then the positive worm lines were transferred to large beakers respectively for expanded reproduction. Sampling test of the reproduction offspring, collect and feed the virus-carrying SBPH after the virus-carrying rate is stable.

2) RSV Inoculation of Transgenic Oryza sativa Overexpressing OsHIR3.

T2 generation of three independent transgenic lines overexpressing OsHIR3 (lines OE6, OE8 and OE12) was selected as the test material, and wild type Oryza sativa was selected as the control, sowed simultaneously. 20-40 healthy seeds were selected for each strain, treated with sterile water, and soaked and germination in an incubator at 37° C. 2 days later, the seeds were exposed and then sown. Each strain was planted in a nutrition bowl (10 cm×10 cm) and cultured in a greenhouse environment.

Plants were transferred to the insect receiving cage, after grew to 3-leaf stage, and the purified 2-4th instar larvae of Laodelphaxstriatellus with high RSV-carrying rate were transferred to the insect receiving cage with 3 -5 Laodelphaxstriatellus per plant. The transgenic plants and wild type were fed with RSV-infected Laodelphaxstriatellu for 2-3 days in parallel at the same time. During the insect receiving period, make sure that the inoculated rice seedlings were evenly poisoned, and dispersing the insects twice a day. After the feeding was completed, all the Laodelphaxstriatellus were removed, the plants were transplanted to the field for disease investigation and analysis after relieved for 2-3 days in a greenhouse environment.

3) Analysis of Transgenic Oryza sativa Plants Against to RSV

About 4-6 days after RSV inoculation, rice seedlings begin to curl up, seedlings with serious diseases begin to die 8-12 days after RSV inoculation. Systemic leaves with serious diseases appear obvious disease spots and curl up 20 days after RSV inoculation. Subsequently, some plants with serious diseases gradually die, disease spots appear on leaves of plants with strong resistant ability, while some symptoms gradually become cryptogenic as the plants grow.

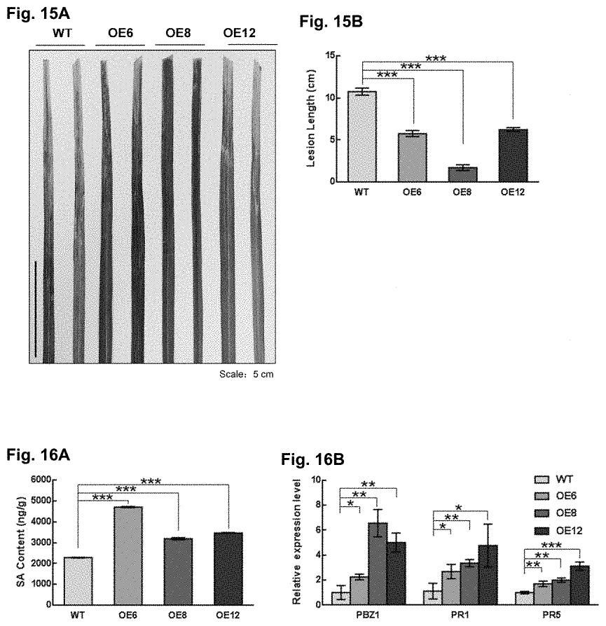

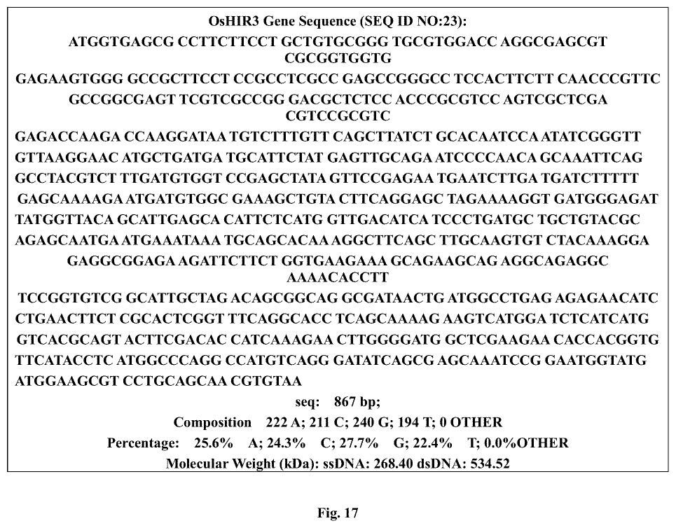

To analyze whether the overexpression of OsHIR3 confer rice plants resistance to RSV,T2 generation of three independent transgenic lines overexpressing OsHIR3 (lines OE6, OE8 and OE12) was selected for anti-RSV identification.30 plants of each line were planted, and wild-type Oryza sativa with same treatment was used as control. Oryza sativa were transplanted into the field after inoculated with RSV, then kept observing and counted the mortality rate continuously. At 10 dpi, approximately 30% of the wild type plants had been dead, while only 10% of transgenic plants from three different transgenic lines had been dead (FIG. 14A). In the surviving plants, RSV symptoms on transgenic plants were significantly milder with fewer yellow stripes on their leaves than on control plants (FIG. 14B). RSV RNAs also accumulated less in transgenic plants according to northern blot analysis (FIG. 14C). The above results showed that overexpression of OsHIR3 effectively reduced the accumulation of RSV RNAs, and OsHIR3 confers plants resistance to RSV.

### Embodiment 14: Analysis of Transgenic Oryza sativa Plants Against to Xoo

1) Xoo Innoculation

The bacterial leaf blight strain P10 (Xoo) was transferred to Cobain's culture solution and cultured in a shaker at 28° C. at 200 r/min for 1 day. The bacterial cells were collected and mixed with ddH2O to OD600 about 0.5. T2 generation of three independent transgenic lines overexpressing OsHIR3 (lines OE6, OE8 and OE12) was selected as the test material, and wild type Oryza sativa was selected as the control, sowed simultaneously and planted in greenhouse. After growing for about 2 months (before heading), they were inoculated with bacterial leaf blight strain P10 by artificial leaf cutting inoculation method. 1-2 weeks after inoculation, the symptom of inoculated Oryza sativa leaves was observed, the length of lesions on diseased leaves of different rice materials was measured, and statistical comparison was made to evaluate Oryza sativa resistance.

2) Measurement of Total Lesion Length

Two weeks after inoculation with XooP10, total lesion length in infected leaves was measured. Statistical results showed that three independent transgenic lines overexpressing OsHIR3(lines OE6, OE8 and OE12) had significantly shorter lesions length than the controls (FIG. 15A). The lesion length of wild type Oryza sativa was (10.5±0.6) cm, while the lesion lengths of the three independent lines OE6, OE8 and OE12 were (5.2±0.5) cm, (2.1±0.3) cm and (6.1±0.2) cm, respectively (FIG. 15B). It therefore appears that the transgenic plants also gained resistance to Xoo. in summary, OsHIR3-mediated basic resistance not only targets RSV, but it also targets other pathogens, such as Xoo.

### Embodiment 15: OsHIR3 Confer Plants Basic Resistance by Positively Regulating SA Pathway

The positive lines with high expression level of OsHIR3s and basal resistance were selected through the above tests. The SA content and the qRT-PCR analysis of key genes involved in SA pathway were detected in those positive lines. Referring to the qRT-PCR instructions, the steps are shown as below:

| | |
|---|---|
| SYBR Green Realtime PCR Master Mix | 18.0 μL |
| cDNA | 6.0 μL |
| Upstream primer | 3.6 μL |
| Downstream primer | 3.6 μL |
| RiNase-free H2O | 10.8 μL |
| Total volume | 36.0 μL |

The reagents were added to RNase-free EP tube in turn, fully mixed, centrifuged instantaneously, added into 384 holes quantitative plate with 10 μL/hole, coated with membrane, and placed in qRT-PCR machine to react at 95° C. for 5 min, 40 cycles: 95° C. for 20 s→58° C. for 20 s→72° C. for 20 s, 72° C. for 10 min.

The specific primers used for qRT-PCR analysis are shown as below:

| Primer name | Sequence |
|---|---|
| RT-OsActin-f | 5′-GGTATCCATGAGACTACATACAACT-3′ |
| | (SEQ ID NO: 32) |
| RT-OsActin-r | 5′-TACTCAGCCTTGGCAATCCACAT-3′ |
| | (SEQ ID NO: 33) |
| RT-OsPBZ1-f | 5′-CACACTCGACGGAGACGAAG-3′ |
| | (SEQ ID NO: 34) |
| RT-OsPBZ1-r | 5′-GCCATAGTAGCCATCCACGAT-3′ |
| | (SEQ ID NO: 35) |
| RT-OsPR1-f | 5′-TATCCAAGCTGGCCATTGCT-3′ |
| | (SEQ ID NO: 36) |
| RT-OsPR1-r | 5′-TTCTCTGGCTGGCGTAGTTC-3′ |
| | (SEQ ID NO: 37) |
| RT-OsPR5-f | 5′-CGCAACAACTGCACCTACAC-3′ |
| | (SEQ ID NO: 38) |
| RT-OsPR5-r | 5′-GGCTAGGAACGAGACGTTGG-3′ |
| | (SEQ ID NO: 39) |

There was significant more SA in three independent transgenic lines overexpressing OsHIR3(lines OE6, OE8 and OE12) than the wild-type plants (FIG. 16A) and the expression levels of PBZ1(also known as NPR1 in rice), PR1 and PR5 which were key genes involved in SA pathway were also upregulated (FIG. 16B). This indicated that OsHIR3 confer plants basic resistance by positively regulating SA pathway.

This is because after plants infected by pathogens, systemic resistance causes the uninfected parts at the distal end to generate resistance to pathogens, which is called systemic acquired resistance (SAR), and this phenomenon has been confirmed in many models of interactions between plants and pathogens. The typical characteristics of SAR is restricting the growth of pathogens and inhibiting the development of infection symptoms. The role of SA in SAR has been reported many times in plants. The mainstream view is that SA is an important signal molecule in the SAR process, and the accumulation of SA will stimulate SAR response. The high expression level of pathogenesis related protein (PR protein) is an important symbol of SAR reaction. And multiple PR proteins always coordinate together instead of a specific PR protein acting alone to cause SAR reaction. PR protein accumulated in tobacco treated with SA or aspirin and confer resistance to Tobacco mosaic virus (TMV) infection. TMV infection can induce a sharp increase of endogenous SA content of tobacco, and the SA content of resistant varieties is significantly higher than that of susceptible varieties. Neither SA accumulating nor SAR activating will happen in the sid1 and sid2 mutant plant, showing sensitivity to Pseudomonas syringae. Those reports further proved that SA is a key signal molecule in SAR process.

Regulatory protein NPR1 is a key component in SA-mediated signal transduction pathway. NPR1 can induce the expression of PR-1 and other resistant genes, thus enhancing the disease resistance of plants. During pathogens infection, SA level is normal, but SAR cannot be induced in nim1 mutant plants, in which the expression of NPR1 gene is affected, indicating that NPR1 acts on downstream of SA and is a key regulatory factor in SAR signal transduction pathway. Despres et. al found that NPR1 can interact with members of Arabidopsis thaliana TGA family which rich in leucine (bZIP) transcription factors, while NPR1 mutant loses its interaction with TGA2, indicating that NPR1-mediated TGA2 binding is critical to the activation of defense genes. When SAR is induced, NPR1 activated PR-1 gene through interaction with transcription factors in the promoter region of PR gene, indicating that the activity of NPR1 is closely related to the regulation of the expression of PR genes. The four-point mutant of NPR1 blocked SA signal and lost interaction with TGA2 and TGA3. TGA2 and TGA3 are able to bind SA response elements of Arabidopsis thaliana PR-1 promoter, and NPR1 and SA-induced PR-lgenea expression were linked by the TGA transcription factor.

EDS1 and NDR1 are two independent positive regulators locating upstream in SA pathway, since the two proteins locate downstream of two functionally distinct classes of R proteins. EDS1 is a positive regulator of basal resistance to pathogen invasion and R protein-mediated resistance ,And EDS1 is also indispensable for Toll-Interleukin-1 receptor (TIR)-type nucleotide binding-leucine rich repeat (NB-LRR) protein-triggered resistance. EDS1 can interacted with PAD4 AND SAG101, forming a complex in the cytoplasm and nucleus and inducing SA accumulation. SA can also inducing the expression of R, EDS1, PAD4 and SID2 genes through feedback mechanism, boosting SA signal. NDR1 is another positive SA positive regulator acting independently from EDS1. NDR1 is required for resistance induced by many R genes encoding CC-NBS-LRR proteins.

1. A method of producing transgenic plants, comprising the steps of:
inserting NbHIR3.1, NbHIR3.2 or OsHIR3 gene into plant binary plasmid expression vector;
transferring positive plasmid vector into Agrobacterium tumefaciens strain;
transferring Agrobacterium tumefaciens strain carrying target gene into plants, thus the transgenic plants overexpressing NbHIR3.1, NbHIR3.2 or OsHIR3 are produced.

2. The method according to claim 1, wherein the nucleotide sequence of said NbHIR3.1 gene is shown as SEQ ID NO:1.

3. The method according to claim 1, wherein the nucleotide sequence of said NbHIR3.2 gene is shown as SEQ ID NO:2.

4. The method according to claim 1, wherein the nucleotide sequence of said OsHIR3 gene is shown as SEQ ID NO:23.

5. The method according to claim 1, wherein said binary plasmid expression vector comprising following structure: LB-35s PolyA-HPTII-35s promoter-Nos-target gene-35s promoter-RB.

6. The method according to claim 1, wherein said transgenic plants have resistance to virus infection.

7. The method according to claim 6, wherein said virus is selected from the group consisting of Turnip mosaic virus (TuMV), Potato virus X (PVX), and RSV.

8. The method according to claim 1, wherein said NbHIR3.1 and NbHIR3.2 genes are cloned from Nicotiana benthamiana, and said OsHIR3 gene is cloned from Oryza sativa L. ssp. japonica. cv.

9. The method according to claim 8, wherein the sequences of primers used for cloning NbHIR3.1 and NbHIR3.2 genes from Nicotiana benthamiana are shown as SEQ ID NOs:3-4 and SEQ ID NOs:5-6, respectively.

10. The method according to claim 8, wherein the sequences of primers used for cloning OsHIR3 gene from Oryza sativa L. ssp. japonica. cv are shown as SEQ ID NOs:24-25.

11. The method according to claim 1, wherein said transgenic plants are Nicotiana benthamiana or Oryza sativa L.

12. The method according to claim 1, wherein the positive plasmid vector is transferred into Agrobacterium tumefaciens strain by electric shock.

13. The method according to claim 1, wherein the SA level in said transgenic plants is increased significantly.

14. The method according to claim 1, wherein said transgenic plants have basic resistance to virus.

15. The method according to claim 11, wherein said transgenic plants is Oryza sativa L. and target gene has the nucleotide sequence showed as SEQ ID NO:23.

16. The method according to claim 15, wherein OsHIR3 up-regulate SA level in transgenic plants through up-regulating the expression of PBZ1 (also known as NPR1), PR1 and PR5.

17. The method according to claim 15, wherein said transgenic plants are produced by callus induced from rice mature embryos.

18. The method according to claim 11, wherein said transgenic plants is Nicotiana benthamiana and target gene has the nucleotide sequence showed as SEQ ID NO:1 and/or SEQ ID NO:2.

19. The method according to claim 18, wherein NbHIR3.2 or NbHIR3.1 increasing SA content in transgenic plants significantly through up-regulating the expression of its receptor EDS1, NPR1 or PR1 significantly.

20. The method according to claim 18, wherein said transgenic plants are produced by leaf disc method.
